(19) United States
(12) Patent Application Publication
(10) Pub. No.: US 2005/0009160 A1
(43) Pub. Date: Jan. 13, 2005

(54) Novel biological approach for forming low-k dielectrics

(75) Inventors: Amos Chou, Hsin-Chu (TW); Antoine Cheng, Jhubei City (TW)

Correspondence Address: GEORGE O. SAILE & ASSOCIATES, 28 DAVIS AVENUE, POUGHKEEPSIE, NY, 12603, US

(73) Assignee: Taiwan Semiconductor Manufacturing Co.

(21) Appl. No.: 10/615,745

(22) Filed: Jul. 9, 2003

(51) Int. Cl. H01L 21/00; C12P 3/00

(52) U.S. Cl. 435/168; 438/1

(57) ABSTRACT

An intermetal or interlevel low-k dielectric layer is formed of the hardened cell walls of microorganisms that are filled with air or an inert gas. The method of formation comprises the formation on the substrate of a protective and adhesion enhancing layer. A culture medium is then applied to the protective and adhesion enhancing layer and seeded with living microorganisms. After the seeded layer has attained a desired thickness by growth and multiplication of the microorganisms, the medium is dried by air or an inert gas, sacrificing the microorganisms, hardening their cell walls and replacing their intercellular material with the drying medium. Finally, a capping dielectric layer is formed over the mass of cell material.

### BACKGROUND OF THE INVENTION

1. Field of the Invention

This invention relates generally to the fabrication of integrated circuits and, more specifically, to the formation of low-k (low dielectric constant) dielectric material on substrate surfaces having circuitry formed thereon.

2. Description of the Related Art

The fabrication of integrated circuits often requires the formation of electrically active elements that are in such close proximity to each other that there is significant and unwanted capacitative coupling between them. This problem can arise between adjacent lines of metallization on a common plane or between closely formed metallic interconnects or between nearby circuit elements disposed on different levels of a multilevel fabrication. The most effective method for alleviating the problem of unwanted capacitative coupling is to fill the region between the coupled elements with dielectric material of as low a dielectric constant as possible (low-k dielectric material). Air is one of the lowest dielectric constant materials available, but it lacks the necessary strength and structural integrity to perform well as an interlayer or intermetal dielectric. One approach that is used in the prior art is to incorporate air into a dielectric material of higher dielectric constant but of greater structural integrity. This can be done by forming voids in the higher-k material. Leung et al (U.S. Pat. No. 6,204,202 B1) teaches a method for forming porous films of low dielectric constant, specifically nanoporous silica films. These films are produced by mixing a non-volatile thermally degradable polymer with various mixtures of silicon based materials. The resulting mixture is applied to a circuit and the thermally degradable portion of the mixture is subjected to various thermal treatments. As a result of the treatments, the polymer is degraded and voids are left behind Chang et al. (U.S. Pat. No. 6,159,842) teaches a method for fabricating a low-k intermetal dielectric layer by first covering the metal lines with a protective low-k fluorine doped oxide, filling the lined gaps so formed with a porous, spun-on dielectric layer, then covering the entire structure with a more dense dielectric layer to provide the needed structural integrity. Chooi et al. (U.S. Pat. No. 6,265,321 B1) teaches a method for confining air-plugs between metal interconnects. The method forms an “air-bridge,” a porous layer, over a layer containing a reactive organic material that is deposited between the interconnects. An oxygen plasma treatment through the pores of the air-bridge removes portions of the reactive material, leaving air-filled plugs between the interconnects. An early use of air-bridges is provided by Matthews (U.S. Pat. No. 5,171,713). Matthews teaches the fabrication of a multilevel integrated circuit in which successively formed levels are separated by polyimide layers through which interconnects pass. The polyimide is finally vaporized by a plasma ashing process, leaving the interconnects essentially suspended in and surrounded by the air between the layers. All of the prior art cited above essentially teaches the introduction of additional air into regions first filled with materials that have less air content. The present invention accomplishes the formation of an air-filled dielectric material of structural integrity by a novel process involving the organic growth of living cellular biological matter, such as microorganisms. When such matter is killed and dried, the cell walls harden and the interior cellular material is replaced by air. Thus, a single biological process replaces the more complicated processes of the prior art.

### SUMMARY OF THE INVENTION

A first object of this invention is to provide a method for forming a low-k dielectric material having structural integrity.

A second object of this invention is to provide a method for forming such a low-k dielectric material as an intermetal dielectric layer.

A third object of this invention is to provide a method for forming such a low-k dielectric material as an interlayer dielectric.

In accord with the objects of this invention there is provided a method for forming a low-k dielectric material as a layer of organically grown cellular biological material, such as microorganisms. The living biological material layer is grown in a culture medium that is applied to the region on which the layer is desired after first covering the region with a protective dielectric layer which also enhances adhesion. After the living material has grown to a desired thickness, the culture medium is washed out, resulting in the death (sacrifice) of the material, and the material is dried, producing hardened cell wall structures within which air has been confined. A protective dielectric capping layer is then formed over the sacrificed biological layer and planarized by an appropriate polishing method, if required, for the formation of subsequent additional levels.

### BRIEF DESCRIPTION OF THE DRAWINGS

The objects, features and advantages of the present invention are understood within the context of the Description of the Preferred Embodiments, as set forth below. The Description of the Preferred Embodiments is understood within the context of the accompanying figures, wherein:

FIG. 1 is a schematic cross-sectional view of a typical region of a microelectronics fabrication formed on a substrate prior to being covered by an intermetal low-k dielectric layer. The region contains several metal lines and the figure shows the spaces between them and the thin protective and adhesion enhancing dielectric layer formed over them.

FIG. 2 shows the region of FIG. 1 wherein a growth of microorganisms had proceeded to a desired thickness.

FIG. 3 shows the sacrificed microorganisms subsequent to drying out the culture medium and covering them with a dielectric capping layer.

### DESCRIPTION OF THE PREFERRED EMBODIMENT

The preferred embodiment of the present invention provides a method of filling intermetal or interlevel spaces in a microelectronics fabrication with a low-k dielectric material formed of the hardened cell wall structures of microorganisms grown in a culture medium and then sacrificed by the drying out of that medium. The drying process destroys the living intercellular material of the microorganisms, replacing it with air or an inert gas used in the drying process and leaving behind their hardened cell structure to trap that air or inert gas and provide, thereby, a low-k dielectric material with a high degree of structural integrity and stability.

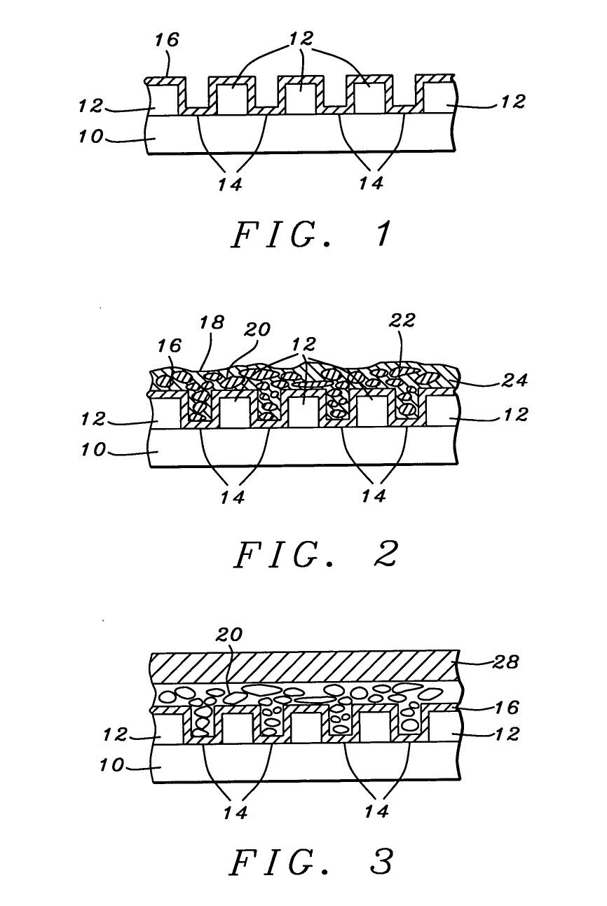

Referring first to FIG. 1, there is shown a schematic cross-sectional view of a portion of a microelectronics fabrication. To simplify the depiction of the process, the portion shown is a single level of such a fabrication, which can be a multilevel fabrication, and is represented by a substrate (10) on which has been formed several lines of metallization (12). Within such a fabrication, the present invention can provide both an intermetal low-k dielectric layer on a single layer or an inter-level dielectric layer between successively fabricated layers. It is also to be noted that the substrate may have a variety of topographic features that extend above and below the substrate surface. The metal lines and substrate have been cleansed of any masking material (eg. photoresistive material) and the lines and substrate surfaces between them (14) are covered by a thin dielectric layer (16), typically a layer of silicon rich oxide in a thickness range between approximately 300 to 2000 angstroms, which may serve multiple purposes within the fabrication, such as a protective layer for the lines, or as a capping oxide for dried cellular growth already present, but it will also serve as an adhesion enhancing layer for the subsequent new microorganism growth.

Referring next to FIG. 2, there is shown the fabrication of FIG. 1 wherein a microorganism infused (seeded) culture medium (18) has been formed over the lines and in the spaces between them. The culture medium is a mixture of nutrients specific to the particular cell-lines of microorganisms being used. Microorganisms preferred for the present process can be coral cell microorganisms, silicon-containing algae or genetic clones of such or similar microorganisms. Typically, the microorganisms will be obtained from cultured cell lines and the culture media will be specific to those organisms. The nature of such cell lines and their appropriate media is well known in the biological arts. The dielectric layer (16) serves to assist the microorganisms in adhering to the fabrication and, at the same time, protects the lines and substrate from adverse activity of the culture medium. The cellular outer layer of the microorganisms, which may be homogeneous or heterogeneous, are shown schematically as ovals (20) filled with living intercellular matter (22), which is shown as a shaded area within the ovals. The culture medium (24) itself, which, as noted above, may be a medium such as a mixture of specific nutrients, is also shown shaded. The microorganisms form a generally matted and unstructured mass, which will ultimately contribute to the strength of the dielectric layer. The microorganisms are allowed to grow and multiply until a predetermined thickness of the layer has been reached.

Referring next to FIG. 3, there is shown the fabrication of FIG. 2 after the desired layer thickness has been achieved and wherein the microorganisms and culture medium have been subjected to drying by means of air or an inert gas. The drying process removes the culture medium, sacrifices the microorganism, hardens their cell walls (20), which are typically rich in silicon based materials and can easily bridge voids and spaces on the substrate, and replaces the intercellular material with either the air or the inert gas (note that the cell walls are now shown empty of intercellular living material). A protective dielectric capping layer (28), which is preferably a layer of silicon rich oxide is formed over the mass of dead cells. The capping layer is typically planarized so that additional levels of microelectronic circuitry can be formed thereon. There are many processes well known in the prior art for producing such a planar surface.

As is understood by a person skilled in the art, the preferred embodiment of the present invention is illustrative of the present invention rater than limiting of the present invention. Revisions and modifications may be made to methods, materials, structures and dimensions employed in the present method for forming a low-k dielectric layer, while still providing a method for forming a low-k dielectric layer, in accord with the spirit and scope of the present invention as defined by the appended claims.

1. A method for forming a low-k dielectric layer comprising:
providing a substrate;
forming on said substrate, by a process comprising the organic growth and sacrifice of living biological material, a low-k dielectric layer of structural integrity.

2. The method of claim 1 wherein the substrate is a microelectronics fabrication having topographic features comprising at least metal lines with spaces between them.

3. The method of claim 2 wherein the substrate is at least partially covered by a protective and adhesion enhancing dielectric layer.

4. The method of claim 3 wherein the dielectric layer is a layer of silicon rich oxide formed to a thickness between approximately 300 to 2000 angstroms.

5 The method of claim 4 wherein said organic growth and sacrifice process further comprises:
applying a culture medium on said substrate;
seeding the culture medium with living microorganisms, said microorganisms having cell walls filled with intercellular material;
allowing the microorganisms to grow and multiply until a predetermined thickness of the microorganism seeded culture medium has been attained;
drying the microorganism seeded culture medium using a drying medium, whereby said microorganisms are sacrificed, said intercellular material is eliminated and is replaced by said drying medium said cell walls are hardened to provide structural integrity and a low-k dielectric layer is thereby formed;
forming a capping layer over said low-k dielectric layer;
planarizing said capping layer.

6. The method of claim 5 wherein said capping layer is a layer of silicon rich oxide formed to a thickness of between approximately 300 to 2000 angstroms.

7. The method of claim 5 wherein the culture medium is formed so as to cover the adhesion enhancing and protective layer and fill the spaces between the metal lines and other topographic features.

8. The method of claim 6 wherein the culture medium is a mixture of specific nutrients for specific cell lines.

9. The method of claim 7 wherein the microorganisms seeding the culture medium are specific cell lines appropriate to the medium.

10. The method of claim 9 wherein the microorganisms are cultures of coral cell lines.

11. The method of claim 9 wherein the microorganisms are cultured from silicon-rich algae lines.

12. The method of claim 9 wherein the microorganisms are cultured from genetic clones of coral cell lines.

13. The method of claim 9 wherein the microorganisms are cultured from genetic clones of silicon-rich algae lines.

14. The method of claim 5 wherein the drying medium is air.

15. The method of claim 5 wherein the drying medium is an inert gas.

16. A microelectronics fabrication having an low-k dielectric layer formed thereon comprising:
a substrate having topographic features comprising at least metal lines with spaces between them.
a protective and adhesion enhancing dielectric layer formed on said substrate;
a low-k dielectric layer formed on said protective and adhesion enhancing dielectric layer, said low-k layer further comprising the skeletal remains of microorganisms wherein the regions within and between said remains contain air or inert gas;
a planarized capping layer, formed over said low-k dielectric layer.

17. The fabrication of claim 16 wherein said adhesion enhancing dielectric layer and said capping layer are layers of silicon rich oxide formed to a thickness between approximately 300 and 2000 angstroms.

18. The fabrication of claim 17 wherein said skeletal remains were formed by drying a nutrient rich culture medium containing living microorganisms that had been applied to said substrate.

19. The fabrication of claim 18 wherein said skeletal remains are the hardened cell walls of said microorganisms.

20. The fabrication of claim 19 wherein the microorganisms are cultures of coral cell lines.

21. The fabrication of claim 19 wherein the microorganisms are cultured from silicon-rich algae lines.

22. The fabrication of claim 19 wherein the microorganisms are cultured from genetic clones of coral cell lines.

23. The fabrication of claim 19 wherein the microorganisms are cultured from genetic clones of silicon-rich algae lines.

24. A multilevel microelectronics fabrication having a low-k dielectric layer formed on each level, said layer comprising:
a protective and adhesion enhancing layer formed on said level;
a low-k dielectric layer formed on said protective and adhesion enhancing layer, said low-k layer being formed of the hardened cell walls of microorganisms and the regions within and between said cell walls being filled with air or an inert gas;
a capping layer, formed over said low-k dielectric layer and planarized.
